(19) United States
(12) Patent Application Publication
(10) Pub. No.: US 2009/0324636 A1
(43) Pub. Date: Dec. 31, 2009

(54) FISH OIL IN STABILIZED FORM

(75) Inventors: John Piechocki, Odenton, MD (US); David J. Kyle, Catonsville, MD (US)

Correspondence Address: MOORE & VAN ALLEN PLLC, P.O. BOX 13706, Research Triangle Park, NC, 27709, US

(73) Assignee: ADVANCED DIONUTRITION CORPORATION, Columbia, MD (US)

(21) Appl. No.: 11/914,355

(22) PCT Filed: May 11, 2006
(86) PCT No.: PCT/US2006/018398

§ 371 Date: Mar. 17, 2009

Related U.S. Application Data
(60) Provisional application No. 60/680,397, filed on May 11, 2005.

(51) Int. Cl. A61K 36/02

(52) U.S. Cl. 424/195.17

(57) ABSTRACT

A method of production and composition of a extremely stable fish oil blend and method of use of said blend. The instant invention provides phospholipid additives to fish oil or other long-chain polyunsaturated fatty acid in an admixture that enhances the stability of the final product. Most preferably said composition utilizes a crude extract of an algal producer of long-chain polyunsaturated acids containing soaps and phospholipids added to fish oil or other purified long-chain polyunsaturated fatty acid to provide a surprisingly stable product.

### BACKGROUND OF THE INVENTION

The invention relates generally to the field of compositions containing fish oil.

Fish oils are known to provide many healthy benefits including offering protection against heart disease, depression, bipolar disease, attention-deficit hyperactivity disorder, alcoholism, postpartum depression and Alzheimer's disease, and they may be useful for diabetes and arthritis as well (3,4,7,9). The key components of fish oil that provide these healthy attributes are the omega-3 very long chain polyunsaturated fatty acids (VLC-PUFAs) which include, but are not limited to, eicosapentaenoic acid (EPA) and docosahexaenoic acid (DHA). The food and feed industries would like to take advantage of these healthy attributes in the preparation of “functional foods” or “functional feeds” by the inclusion of fish oil in conventional food products. However, a common problem associated with the use of fish oil in food or feed applications is the rapidity with which the oil oxidizes and goes rancid, leading to negative organoleptic characteristics and rejection of the product by the consumer (man or other animals) (5,6). Not only is this a problem with respect to the taste of the supplemented product, but oxidized fish oil can also contain chemical moieties that are toxic (e.g., lipid hydroperoxides) to the human or other animal.

Various attempts have been made to stabilize fish oils through the use of lipophilic antioxidants (e.g., Vitamin E or various organic quinones) or by setting up a physical barrier between the fish oil and the food product matrix (e.g., through microencapsulation and/or coating). However, each of these solutions has significant limitations. In many cases the best antioxidants (e.g., TBHQ or ethoxyquin) have been banned for use in foods due to low-level chemical toxicity or carcinogenicity. Other natural antioxidants (e.g., Rosemary extract) are very expensive and are not as effective as the synthetic antioxidants. Microencapsulation of fish oil is expensive and may not be compatible with certain food matrices as most encapsulation materials are not water stable. It has been previously shown that algal oils and fish oils can be stabilized by the addition of soy lecithin when added in large quantities (i.e., in a ratio of 1:4; lecithin to oil) (5,10). Soy lecithin does not contain any of the VLC-PUFAs, which are known to accelerate oxidation, and the required quantities result in the dilution of the VLC-PUFA level of the final mixture.

Certain algal oils are also known to be highly enriched in omega-3 VLC-PUFAs. In the case of certain fermentatively produced algae rich in omega-3 VLC-PUFAs (e.g., Crypthecodinium cohnii and Schizochytrium sp.), the process has been scaled up and commercialized (2,8,11,12). This process involves growing the algae, stressing the organisms to induce the production of an oil, harvesting and drying the algae, extracting the oil with an organic solvent, and purifying the oil using processes well known in the art for vegetable oil processing. The overall process, as applied to these algae, results in the production of a fine, bright oil highly enriched in omega-3 VLC-PUFAs. These oils, like fish oil, are not as stable as other vegetable oils such as corn oil, soybean oil, canola oil, palm oil, etc., because they are enriched in VLC-PUFAs that are inherently susceptible to oxidative breakdown. Indeed all oils rich in VLC-PUFAs are exceptionally susceptible to oxidation.

### BRIEF SUMMARY OF THE INVENTION

The present invention is based on the inventors' discovery that extracts from algae rich in VLC-PUFAs are remarkably, and unexpectedly resistant to oxidative breakdown, and that when admixed with fish oil, refined algal oil, or the algal biomass itself, these extracts have the effect of stabilizing all the VLC-PUFAs in the mixture. Such a newly stabilized form of fish oil can be used as a food additive for products intended to be consumed by animals including man, thereby solving a major problem of maintaining the VLC-PUFAs in the product with a minimum of oxidative damage. Furthermore, because of the contribution of the VLC-PUFAs from the extract itself, this problem is solved without significant dilution of the active VLC-PUFAs themselves. Quite unexpectedly, the inventor discovered that the addition of a DHA-rich extract of Crypthecodinium cohnii, an alga that is highly enriched in DHA oil, conveys a significant stabilization effect on fish oil.

### BRIEF SUMMARY OF THE SEVERAL VIEWS OF THE DRAWINGS

FIG. 1 is a flow sheet showing diagrammatically the processing of Crypthecodinium cohnii fermentatively grown cells into fractions useful for the instant invention. The alga is grown fermentatively on glucose/yeast extract based medium, harvested by centrifugation to produce a wet biomass that could be used directly to mix with high VLC-PUFA material to stabilize. Alternatively, the pelleted cells are spray dried to produce a powder. This powder is then extracted to produce a crude algal extract that can be used in the instant invention. The residual material left behind after extraction is also used in the current invention, the Biomeal. This can be further refined to produce an oil and a triglyceride-depleted fraction, the crude algal extract also described in the current invention.

### DETAILED DESCRIPTION OF THE INVENTION

This invention relates to the preparation of a stabilized form of fish oil or other unstable edible oil, the composition of said oil, and its use in food and feed applications. The composition comprises a combination of fish oil and an extract of algae rich in omega-3 very long chain polyunsaturated fatty acids (VLC-PUFAs) including, but not limited to, docosahexaenoic acid (DHA) and/or eicosapentaenoic acid (EPA), in a ratio of about 0.01 to 100 parts algal extract to one part fish oil. The stabilized fish oil composition of the invention is used as an ingredient to provide a functional food for animals, including man, wherein the VLC-PUFAs provide their clinical effect with improved resistance to oxidative deterioration in the food product.

Another embodiment of this invention relates to the use of the extract of algae rich in omega-3 VLC-PUFAs such as, but not limited to, Crypthecodinium, Schizochytrium, Thraustochytrium, and Ulkenia species as an additive to foods for animals, including man, to provide a highly stabilized form of VLC-PUFAs in the food product.

Another embodiment of this invention relates to the use of an extract of algae rich in omega-3 VLC-PUFAs such as, but not limited to, Crypthecodinium, Schizochytrium, Thraustochytrium, and Ulkenia, as an antioxidant. This extract can be added back to an algal biomass to prevent excessive oxidation during processing or storage of said biomass. This extract can also be added directly to a food product to provide a stable source of omega-3 VLC-PUFAs.

Another embodiment of this invention relates to the use of Crypthecodinium, Schizochytrium, Thraustochytrium, and Ulkenia algal biomass or Biomeal added directly to fish oil wherein the resulting slurry has improved stability compared to the oil itself. The resulting stabilized mixture can be used as a food product to deliver stable omega-3 VLC-PUFAs to animals in feeds or food products.

### DEFINITIONS

As used herein, each of the following terms has the meaning associated with it in this section.

“VLC-PUFA” refers to a long chain polyunsaturated fatty acid with a carbon chain length of 20 or greater and 3 or more double bonds. These can be, but are not limited to, docosahexaenoic acid (DHA), docosapentaenoic acid (DPA), arachidonic acid (ARA), and eicosapentaenoic acid (EPA).

“Crude Algal Extract” refers to any material extracted from an algal biomass using organic solvents such as, but not limited to hydrocarbons (e.g., hexane, pentane, etc.), alcohol (e.g., isopropanol, ethanol, etc.), halogenated hydrocarbons (e.g., chloroform, methylene chloride, etc.), or supercritical gases (e.g., carbon dioxide, propane, etc). Additionally, non-traditional extraction processes utilizing aqueous or dry extraction that provide equivalent Crude Algal Extracts are included in this definition.

“Algal extract” refers to the residual material following a refining step (physical or chemical) that removes a portion of the triglyceride contained in the Crude Algal Extract.

“Biomeal” refers to the solid material remaining after oil extraction. This consists of cell walls, proteins, residual oils, stabilizing compounds, and other compounds that resist the extraction process or are precipitated during processing of the oil.

“VLC-PUFA Algae” refers to any algal species that produces VLC-PUFAs as defined above. Examples of such VLC-PUFA Algae are, but not limited to, Crypthecodinium cohnii, Crypthecodinium sp., Schizochytrium sp., Porphyridium cruentum, Phaeodactylum tricornutum, Neochloris sp., Nitzichia alba, and Cyclotella sp.

“Marine Oil” refers to any oil, which is predominantly triglyceride, that has been extracted from an aquatic animal (marine or fresh water) including fish (e.g., Catfish, Menhaden, Salmon, Tuna, shark, etc), mammals (e.g., whale, seal, etc), crustaceans (e.g., shrimp, crabs, etc.), cephalopods (e.g., squid, cuttlefish, etc.), and mollusks (e.g., oysters, clams, etc.).

### DETAILED DESCRIPTION

In accordance with the present invention, a composition has been developed comprising a marine oil and a Crude Algal Extract or and Algal Extract containing VLC-PUFAs which exhibits an unexpectedly high degree of oxidative stability.

Microalgae to be used in this invention contain VLC-PUFAs and can be grown photosynthetically or harvested from a photosynthetic culture, pond or raceway. Not all algae contain VLC-PUFAs, but those that do are well known as their fatty acid compositions have been published. Such algae would include, but not be limited to, green algae (e.g., Nannochloris, Nannochloropsis, and etc.), Chrysophytes (e.g., Isochrysis, and etc.), Diatoms (e.g., Cyclotella, Navicula, Tetraselmis, and etc.), Phaeophytes (e.g., Laminaria, Ulva, and etc.). Alternatively, and preferably, the algae can be grown by fermentation. Such algae are also well known and would include, but not be limited to, Dinoflagellates (e.g., Crypthecodinium, etc) and chytrids (e.g., Schizochytrium, Thraustochytrium, Ulkenia, etc). These latter algae contain little or no chlorophyll, which significantly improves the stabilization effect of the Crude Algal Extract and Algal Extract. Presumably, algae enriched in VLC-PUFAs have evolved antioxidant mechanisms to protect these VLC-PUFAs from oxidative breakdown in situ, which could lead to cell death.

Microalgae are harvested by any standard means including centrifugation, flocculation, filtration, etc. and the concentrated algae are used as the starting material for the preparation of the Crude Algal Extract. Algae can be dried prior to extraction or they can be extracted as a wet paste. Algal oils are typically extracted using organic solvents such as hexane, alcohol, or any other solvents used for vegetable oil extraction. The Crude Algal Extract can be used directly in this invention or it can be further purified by standard vegetable oil refining steps that include degumming, refining, bleaching and deodorizing. The process waste stream, generated by the refining of the Crude Algal Extract, is particularly useful in this invention. The Algal Extract is an oily or greasy material that is highly enriched in the VLC-PUFA of the algae in the form of triglyceride, free fatty acid, fatty acid soaps, and phospholipid. If photosynthetic algae have been used, the chlorophyll must be removed (e.g., by bleaching) to improve the stabilization characteristics of the extract. Fermentatively grown algae such as Crypthecodinium or Schizochytrium, do not contain any chlorophyll and this step is not necessary.

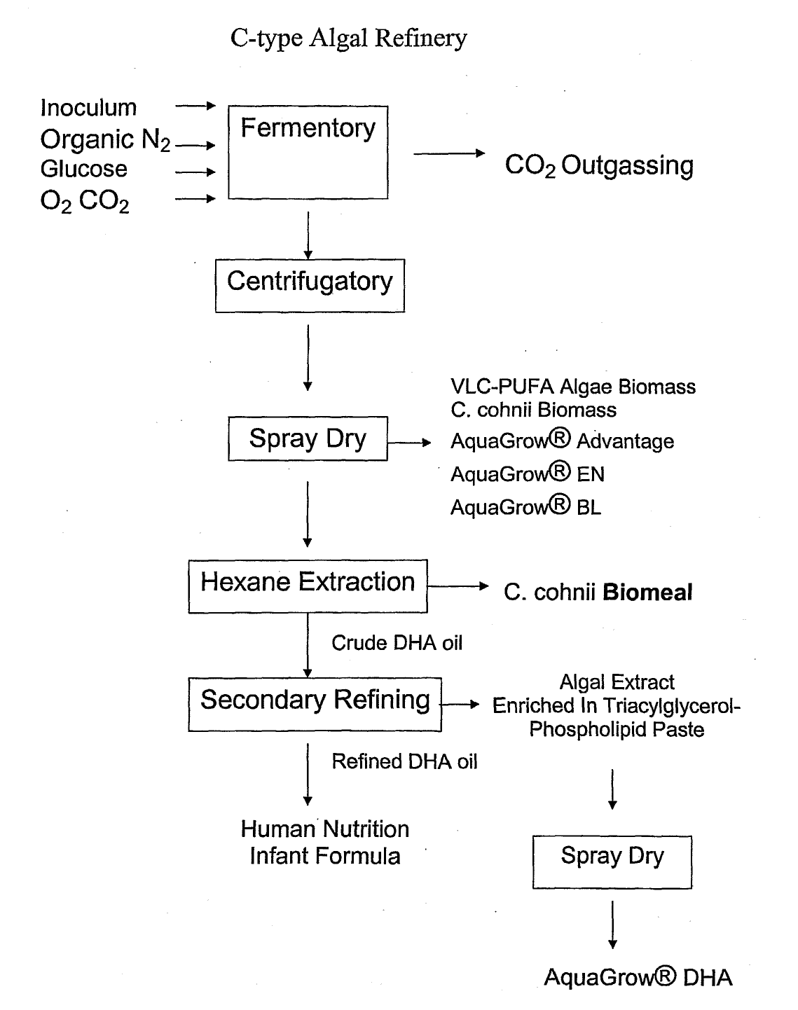

Both physical and chemical refining processes can be used to produce the Algal Extract. Chemical refining processes typically involve washing the crude oil extract with alkali and phosphoric acid and then separation of the more polar lipid components (e.g. free fatty acids, soaps, phosphatides, and etc.) from the triglyceride-rich micellar fraction. An example of the processing that could lead to fractions useful for the present invention is provided for the processing of Crypthecodinium biomass (FIG. 1).

The Crude Algal Extract or the Algal Extract is then added directly to the marine oil and blended using any conventional mixer such as but not limited to a high speed disperser, gear drive drum mixer, double blade planetary mixer, or shaft & propeller mixer. The resulting composition is still highly enriched in EPA and DHA and has stability far greater than the marine oil alone. The resulting mixture can be further stabilized with the addition of other known antioxidants such as, but not limited to, vitamin E, carotenoids, gallates, lecithin, quinines, etc.

The resulting stabilized marine oil can be used directly in a food or feed product by admixing or top coating the product with the oily material by any method known in the art. Alternatively, the resulting stabilized marine oil can be processed into a dry powder form by combining it with other carbohydrate polymers (e.g., soluble or nonsoluble starch, cellulose, chitosan, etc.) or proteins (e.g., gelatin, whey protein, casein, etc.). The resulting admixture can be used directly or encapsulated, extruded, expanded, or processed in any way suitable for use as a food or feed product.

In another embodiment of this invention the Crude Algal Extract or the Algal Extract is added directly to any food matrix with or without marine oil to provide a stabilized form of the VLC-PUFAs in the food product. The Crude Algal Extract or the Algal Extract can be blended directly with the dry ingredients of processed or unprocessed grains prior to the manufacturing of cereals or cereal-based products such as food bars. The Crude Algal Extract or the Algal Extract can also be blended directly with the aqueous or emulsified ingredients in the preparation of margarines, spoonable dressings, pourable dressings, etc. prior to blending, mixing, processing and packaging.

The Crude Algal Extract or the Algal Extract can also be added directly to unextracted algal biomass by top coating the dried biomass or adding the liquid extract to the algal paste prior to the drying step. Such a process will add significant stability to the biomass itself once dry so that artificial antioxidants such as ethoxyquin need not be used. Lecithin can also be added to the algal biomass by spray-coating the dried biomass with a diluted lecithin mixture or by adding the lecithin directly to the algal paste prior to the drying step. Lecithin can be combined with the Crude Algal Extract or the Algal Extract for improved antioxidant capacity or used alone to stabilize the biomass.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

### Preparation of Crude Algal Extract

An algal oil extract is prepared by first growing the algal Crypthecodinium cohnii in a fermentor with a medium comprising glucose, yeast extract and artificial seawater as described by Kyle (U.S. Pat. No. 5,407,957). The algal biomass is harvested by a continuous flow centrifugation and the solids are dried with a spray dryer. Dried solids are extracted with hexane and the Crude Algal Extract in hexane (micella) is separated from the extracted algal meal by centrifugation. The hexane is then removed from the micella leaving Crude Algal Extract. The Crude Algal Extract is then refined by the addition of aqueous alkali and centrifugation. The aqueous layer (Algal Extract) contains a mixture of triglyceride oils, free fatty acids, soaps, and phospholipids in an aqueous emulsion. This material is commercially available from Advanced BioNutrition Corp. and represents the starting point for this invention.

### Example 2

### Preparation and Analysis of an Algal-Stabilized Fish Oil

The Crude Algal Extract of Example 1 was mixed vigorously with fish oil (Omega Protein; Houston, Tex.) in various ratios and the resulting mixtures were tested for oxidative stability by measuring the peroxide value (PV) after exposure of the samples to 0 h at ambient temperature (about 22° C.), 4 h or 20 h at 97.8° C. with air flow of 2.33 milliliters/second. Samples were prepared such that the dry product was 40% fat. For each sample the Miramist 662 (Staley, Ind.), Star dri (Tate & Lyle, UK), fish oil and DHA extract were weighed into a 2 L beaker. Distilled water was added to bring the final volume to 1 L. The mixture was homogenized using a Braun hand blender. Samples were frozen and subsequently freeze dried. New Jersey Feed Laboratory (Trenton, N.J.) performed the PV analyses (Table 1). Total oxidative stability was achieved by a ratio of 1.4:0.6 (fish oil:DHA extract (fat)). This is equal to 70% fat from fish oil and 30% fat from DHA extract. Because the DHA extract is not all fat it takes exactly a 1:1 ratio of the raw DHA extract to fish oil to achieve the desired fat levels. If we assume that fish oil is 10% DHA and that 50% of the fat in the DHA extract is DHA then the ratio of 1.4:0.6 fish oil fat to DHA extract fat will yield a dry product that contains approximately 8.8% DHA (by weight) (given the formulation listed above). If we assume that fish oil is 10% DHA and that 50% of the fat in the DHA extract is DHA than the ratio of 1.4:0.6 fish oil fat to DHA extract fat will yield a dry product that contains approximately 8.8% DHA (by weight) (given the formulation listed above)

TABLE 1
Peroxide values of Algal Extract/Fish Oil at various Admix
Ratios in accelerated degradation study.

| Sample | | | | | Formulation (in grams) | | | |
|---|---|---|---|---|---|---|---|---|
| (fish oil:DHA | | PVs (mep/kg) | | | | Star | Fish | DHA |
| extract*) | | Initial | 4 h | 20 h | Miramist | dri | oil | Extract |
| 1 | 1 | 1 | 4.4 | 8.8 | 130 | 170 | 100 | 238 |
| 1.2 | 0.8 | 1.4 | 4.2 | 9.6 | 130 | 170 | 120 | 190 |
| 1.4 | 0.6 | 0.8 | 2.2 | 3.8 | 130 | 170 | 140 | 143 |
| 1.6 | 0.4 | 3.6 | 15.8 | 26.6 | 130 | 170 | 160 | 95 |
| 1.8 | 0.2 | 3 | 24.2 | 48 | 130 | 170 | 180 | 48 |
| 2 | 0 | 3.6 | 175.8 | 150 | 130 | 170 | 200 | 0 |

*This number represents only the fat in the DHA extract and not the extract itself. The extract also contains carbohydrate, protein and water that have been accounted for in the formulations.
*The “DHA Extract column indicates the amount of actual extract required to achieve the fat:fat sample ratios.

### Example 3

### Use of an Algal-Stabilized Fish Oil in Yoghurt

A fish oil (Menhaden oil) and algal extract (from C. cohnii) is prepared according to Example 1 at a ratio of 1 part algal extract to 3 parts Menhaden oil. This mixture is then added to yoghurt to provide a DHA level of 100 mg per serving using a high shear mixer and blended until smooth and uniform. The functionalized yoghurt is then packaged into plastic containers and sealed.

### Example 4

### Use of an Algal-Stabilized Fish Oil in a Food Bar

A fish oil (Tuna oil) and algal extract (from Schizochytrium Sp.) is prepared according to Example 1 at a ratio of 1 part algal extract to 1 parts tuna oil. This mixture is then added to a cereal bar mixture comprising granola, corn syrup, crisp rice, raisins, oatmeal, sorbitol, salt, natural flavors, cultured whey, molasses, citric acid and water at a level corresponding to 200 mg DHA per serving (28 g), mixed with a high shear mixer, and extruded at a temperature of 100° C. This is followed by drying in an oven at 130° C., where the product temperature is a maximum of 115° C. The cereal food bars are then cut into individual portions of about 28 g and sealed in foil packages.

### Example 5

### Use of an Algal-Stabilized Fish Oil to Topcoat a Feed Pellet

A fish oil (Menhaden oil) and algal extract (from C. cohnii) is prepared according to Example 1 at a ratio of 1 part algal extract to 5 parts Menhaden oil. This mixture is then transferred to a holding tank and maintained in a liquid state. This liquid mixture is heated to about 50 C and spray coated onto standard feed pellets to provide a composition that contains about 1% by weight DHA. These feed pellets are then packaged and can be stored for several months prior to use.

### Example 6

### Production of an Algal-Stabilized Fish Oil in an Extruded Pellet

A fish oil (Menhaden oil) and algal extract (from C. cohnii) is prepared according to Example 1 at a ratio of 1 part algal extract to 1 part Menhaden oil. This mixture is then added to a combination of wheat flour (80%), alfalfa meal (15%), and molasses (5%) and loaded into a twin screw extruder and extruded into pellets of 5 mm in length by 3 mm in diameter. The resulting extruded product contains about 20% oil and the oil contains about 20% DHA (4% DHA by weight of the final product).

### Example 7

### Use of an Algal-Stabilized Fish Oil Premix as an Ingredient in a Feed

Algal Extract stabilized fish oil pellets are prepared according to Example 5. These pellets are admixed with a conventional swine feed at a level of about 5% of the total feed. The resulting mixture contains about 1% DHA and is fed to pregnant and nursing sows to improve the DHA transfer to the fetuses and suckling piglets.

### Example 8

### Stabilization of Algal Biomass with Algal Extract

An algal extract (from C. cohnii) is prepared according to Example 1 or purchased from Advanced BioNutrition Corp. Schizochytrium biomass is produced by fermentation according to U.S. Pat. No. 5,130,242. The Schizoclytrium biomass can be harvested by centrifugation or the entire fermentation broth drum dried, and the Algal Extract is blended with this pellet at a level of 1 part Algal Extract (wet weight) to 20 parts Schizochytrium biomass (wet weight at 10-15% solids). The resulting mixture is then spray dried.

### Example 9

### Stabilization of Algal Biomass with Algal Extract

An algal extract (from Schizochytrium) is prepared according to Example 1 or purchased from Advanced BioNutrition Corp. Schizochytrium biomass is produced by fermentation according to (1,2). The Schizochytrium biomass is harvested by centrifugation and dried with a rotary vacuum dryer. When the Schizochytrium biomass has a solids content of 80%, the Algal Extract is sprayed onto the drying biomass and blended prior to the final stages of drying to less than 8% moisture content. The resulting mixture is a stabilized form of Schizochytrium biomass.

### Example 10

### Stabilization of Algal Biomass with Lecithin

Schizochytrium biomass is produced by fermentation according to (1,2). The Schizochytrium biomass is harvested by drying the entire contents of the fermentation but adding lecithin (Degussa) onto the drying biomass and blended prior to the final stages of drying to less than 8% moisture content. The resulting mixture is a stabilized form of Schizochytrium biomass.

### Example 11

### Stabilization of Fertillium™ with a Mixture

Several formulations of United Feeds' extruded fish oil product Fertillium were made at American Custom Drying Co (Burlington, N.J.). Batch 1 (FO) was a control made following United Feeds' normal Fertillium formulation with fish oil containing ethoxyquin. Batch 2 was made with a mixture of fish oil and DHA extract (FO+DHA). Batch 3 was made with DHA extract only. Initial PV measurements were made following the production. PV measurements were repeated at 4 months and with the following results:

| | Fertillium Mix | PV initial | PV 4 months |
|---|---|---|---|
| Batch 1) | FO | 3.8 | 30.0 |
| Batch 2) | FO + DHA | 0.6 | 7.2 |
| Batch 3) | DHA | | 4.0 |

* Peroxide value or PV reported in mep/kg; all analysis done at New Jersey Feed Labs (Trenton, NJ).

The formulations were calculated to achieve total fat of 40% in a spray dried powder. The FO+DHA blend was formulated such that 20% of the fat was from fish oil and 20% of the fat was from the DHA extract.

The DHA extract clearly adds a significant measure of stability to the fish oil and extends the shelf life of the Fertillium product. Studies are planned for monitoring of stability of these materials on a monthly basis. Keep in mind that a PV of 40 can have harmful effects in animals at high enough doses.

### Example 12

### Use of Algal Biomeal to Stabilize Fish Oil

Fish oil is mixed with Biomeal (Martek Biosciences Corp.) that remains from the production of DHASCO™ (Martek Biosciences Corp.) from Crypthecodinium cohnii. The Biomeal is added at a ratio of 1 part Biomeal to 10 parts oil to form a viscous slurry. This slurry is then incorporated into any standard food preparation to deliver a stabilized DHA.

### Example 13

### Use of Algal Biomass to Stabilize Fish Oil

Fish oil is mixed with freeze-dried algal biomass containing high levels of DHA (Crypthecodinium cohnii; Martek Biociences Corp.). This material is mixed in a ratio of around 2 parts biomass to 10 parts fish oil. This provides a mixture where the DHA level is increased in relation to the EPA level of regular fish oil and a higher level of stability is achieved through the use of the co-mixture of the instant invention.

### Example 14

### Preparation of Crude Algal Extract from Green Algae

The green alga Nannochloropsis is grown phototrophically using Erdschreiber medium (15) in a photobioreactor as described in (14). The algae is extracted as described in Example 1. Crude algal extract, algal extract, biomass, and Biomeal is used as described in previous examples.

### Example 15

### Preparation of Crude Algal Extract from Chrysophytes

The chrysophyte Isochrysis LB987 (Univ. Texas culture collection) is grown phototrophically using Erdschreiber medium (15) in a photobioreactor as described in (14). The algae is extracted as described in Example 1. Crude algal extract, algal extract, biomass, and Biomeal is used as described in previous examples.

### Example 16

### Preparation of Crude Algal Extract from Photosynthetic Diatom

The diatom Cyclotella 1269 (UTex collection) is grown phototrophically in Porphyridium medium (in 500 milliliters combine 200 milliliters glass distilled water, 250 milliliters pasteurized filtered seawater, 50 milliliters soil water supernatant, 0.5 gram yeast extract, 0.5 gram tryptone) in a photobioreactor as described in (14). The algae is extracted as described in Example 1. Crude algal extract, algal extract, biomass, and Biomeal is used as described in previous examples.

### Example 17

### Preparation of Crude Algal Extract from Heterotrophic Diatom

The diatom Cylindrothecia fusiformis (13) is grown heterotrophically using glucose as a carbon source essentially as described in Example 1 for Crypthecodinium. The algae is extracted as described in Example 1. Crude algal extract, algal extract, biomass, and Biomeal is used as described in previous examples.

### Example 18

### Preparation of Crude Algal Extract from a Phaeophyte

Laminaria is collected from the ocean as described in the literature. This material is then extracted as described except that the algal biomass is air dried and a milling step is added after drying. The algae is extracted as described in Example 1. Crude algal extract, algal extract, biomass, and Biomeal is used as described in previous examples.

### Example 19

### Preparation of Crude Algal Extract from the Chytrid Ulkenia

Ulkenia is grown fermentatively and extracted as described in Example 1. Crude algal extract, algal extract, biomass, and Biomeal is used as described in previous examples.

### Example 20

Algal cell biomass (Schizoclzytrium sp.) was cultured by fermentation according to Barclay (U.S. Pat. No. 6,451,567), treated with a natural antioxidant package consisting of vitamins C, Vitamin E, and rosemary extract, harvested by centrifugation and drum dried. This is referred to as INT biomass and was provided by Martek BioSciences Corp (Columbia, Md., USA). The total lipid content of the INT biomass was 59.9% by weight and it was dry blended with 4% by weight deoiled, powdered soy lecithin (Epikuron 100P). The resulting material was placed in an oven set at 100° C. (accelerated stability challenge) and samples were removed every 4 hours and tested for peroxide value (PV). DHA content was also measured at time 0 and 24 hours by gas chromatography of fatty acid methyl esters prepared from the biomass samples. The resulting data are shown in Table 2.

TABLE 2
INT Biomass and INT Biomass + 4% Lecithin
Cooked in a Drying Oven at 100° C.

| | INT | | INT + Lecithin | |
|---|---|---|---|---|
| Time | Peroxide Value | DHA | Peroxide Value | DHA |
| (hrs.) | (kg/kg) | (% of fat) | (kg/kg) | (% of fat) |
| 0 | 7.8 | 34.7 | 5.4 | 30.89 |
| 8 | 13.2 | | 3.2 | |
| 12 | 122.0 | | 6.0 | |
| 16 | 78.9 | | 19.6 | |
| 20 | 17.5 | | 22.0 | |
| 24 | 36.0 | 28.0 | 30.0 | 30.52 |

### Example 21

INT biomass was prepared as in Example 20. Lecithin (Epikuron 100P) was dry blended with the INT biomass at a level of 5% by weight. In a second sample, a commercial antioxidant package provided by Kemin Industries (Des Moines, Iowa, USA) was dry blended with the INT biomass at a recommended level of 1% by weight. The Kemin antioxidant package is a proprietary blend of natural and synthetic antioxidants (excluding ethoxyquin) that is currently being used at a 1% inclusion level to preserve fishmeal. Samples were placed in an oven set at 100° C. for 16 hr in an accelerated stability test. After 16 hr, all samples were removed and the peroxide values (PVs) were determined (Table 3).

### Example 22

INT biomass was prepared as in Example 20. To a 100 g fraction of the biomass, 5 g of lecithin (Epikuron 100P) was added by dry blending. To a second 100 g fraction of biomass, 150 ml of water was added (to mimic the concentrated slurry from a fermentation harvest) and 5 g of Epikuron 100P was added. The resulting slurry was mixed vigorously and freeze dried to remove the water. Samples of dry and wet blended material were placed in an oven set at 100° C. for 16 hr in an accelerated stability test. After 16 hr, the samples were removed and the peroxide values (PVs) were determined (Table 3).

TABLE 3
Peroxide Value of Samples Stabilized With Lecithin or a Commercial
Antioxidant Package Following 16 hr of Treatment at 100° C.

| Sample | Time (hrs.) | Peroxide Value (kg/kg) |
|---|---|---|
| INT | 0 | 5.8 |
| | 16 | 88.0 |
| INT + Kemin package | 16 | 9.8 |
| INT + Lecithin | 16 | 7.6 |
| INT + Lecithin + water and | 16 | 5.4 |
| freeze dried | | |

### Example 23

Various types of lecithins were tested for their ability to stabilize polyunsaturated fatty acid rich material. Samples of fish oil (10 g per 15 ml test tube) were prepared and different types of lecithin were added to the fish oil samples at a 5% inclusion level. In addition, two synthetic antioxidant mixtures were added to the fish oil samples at the recommended 0.1% inclusion level. All samples were left open and the 15 ml test tubes were incubated in a drying oven at 100° C. for 8 hours and sampled every two hours for PV determination (Table 4).

TABLE 4
Peroxide Value of Samples Stabilized With Lecithin or
a Commercial Antioxidant Package Following 16 hr
of Treatment at 100° C.

| | | | | T = 6 hr | T = 8 hr |
|---|---|---|---|---|---|
| | T = 0 hr | T = 2 hr | T = 4 hr | PV- | PV- |
| | PV-Initial | PV-Initial | PV-Initial | Initial | Initial |
| Sample | (meq/kg) | (kg/kg) | (kg/kg) | (kg/kg) | (kg/kg) |
| Control (untreated | 3.6 | 6.0 | 10.0 | 15.2 | 20.4 |
| fish oil) | | | | | |
| ADM | 2.4 | 3.6 | 4.4 | 4.4 | 6.0 |
| Epikuron 100P | | | | | |
| Leciprime 400 | 2.0 | 3.2 | 4.0 | 3.6 | 4.0 |
| Leciprime 1000 | 2.0 | 2.4 | 2.8 | 4.0 | 4.4 |
| ALC Alcoec | 3.2 | 4.4 | 5.6 | 7.8 | 8.1 |
| LV-30 | | | | | |
| Yelkin 1018 | 2.3 | 2.4 | 3.2 | 4.4 | 4.8 |
| Alcolec Xtra-A | 2.8 | 4.4 | 5.6 | 8.0 | 10.0 |
| ADM Nutreon* | 6.4 | 6.8 | 8.1 | 11.2 | 16.4 |
| Kemin Pet-Ox* | 4.4 | 7.2 | 9.6 | 13.6 | 18.4 |

### Example 24

Fish oil samples were set up as in Example 24 and the stabilization effect of only the Yelkin 1018 liquid soy lecithin was tested at different levels of addition from 2% to 8% by weight. The oxidative stability of the oil was determined by analysis using a Rancimat (Metrohm A G, Herisau, Switzerland) and a procedure similar to an Oxidative Stability Index (OSI) determination. This process is a standard method published by the American Oil Chemists Society (AOCS). At various time points during the forced oxidation of the oil, a sample was withdrawn and the peroxide value (PV) was determined (Table 5).

TABLE 5
Peroxide Value of Fish Oil as a Function of
Time in a Rancimat Where the Fish Oil was Stabilized
with Various Amounts of Lecithin.

| | PV - Initial | PV-4 hr | PV-20 hr |
|---|---|---|---|
| % Lecithin | (kg/kg) | (kg/kg) | (kg/kg) |
| 2 | 3.6 | 8.0 | 30.0 |
| 4 | 2.8 | 3.4 | 3.8 |
| 6 | 2.8 | 3.0 | 2.6 |
| 8 | 2.0 | 1.4 | 1.0 |

### Example 25

Algal cell biomass was prepared according to Example 20 and used in the preparation of a feed. The INT biomass was blended with wheat flour, alfalfa meal, and flax meal at a 45% inclusion rate and a second sample was blended at a 32% inclusion rate. In both cases, lecithin (Epikuron 100P) was added at 5% of total diet and the resulting mixture was steam extruded (100° C. for 3 minutes followed by hot air drying for 20 minutes at a final temperature of 145° C.) to form the feed pellets highly enriched with the algal cell biomass. Samples of the extruded products were placed in standard bags and stored at room temperature. After 0, 3 and 7 months the fat was extracted from sample aliquots and the peroxide values (PV) of the extracted fat samples were determined (Table 6).

TABLE 6
Long-term Stability Results of Feeds Highly Enriched
in Algal Cell Biomass Stabilized with Lecithin.

| | Peroxide Value | Peroxide Value | Peroxide Value |
|---|---|---|---|
| Inclusion Rate | 0 Months (kg/kg) | 3 Months (kg/kg) | 7 Months (kg/kg) |
| 45% | 6.4 | 4.0 | 2.2 |
| 32% | 5.4 | 3.2 | 3.2 |

### Example 26

Algal cell biomass was cultured according to Example 20, but freshly harvested biomass was mixed with lecithin and spray dried. Yelkin liquid soy lecithin was added directly to a concentrated fermentation broth of Schizochytrium (ca. 20% solids) at a level of 5% of the solids. This sample and a control sample to which no lecithin was added, were dried in a pilot scale spray dryer (American Custom Drying Co., Burlington, N.J., USA). Fifty-gram samples were transferred to sample containers and stored at either 22° C. or 40° C. The initial peroxide values obtained from oil extracted from the biomass (Table 7) indicate that the addition of lecithin significantly reduced (50%) the oxidation damage due to the spray drying. Initial DHA levels determined by gas chromatography of fatty acid methyl esters prepared from biomass samples also indicated that the addition of lecithin significantly reduced the oxidation damage (loss of DHA) due to the spray drying.

TABLE 7
Peroxide Values from Oil Extracted from Algal Biomass Samples
With and Without Lecithin Following Spray Drying.

| | | DHA level mg/g |
|---|---|---|
| DHA-S Biomass, T = 0 (Initial) | PV (meq/kg oil) | biomass |
| Control (w/o lecithin) | 8.1 | 176.3 |
| with Lecithin | 4.6 | 168.8 |

References cited herein are indicated by the following numbers.
1. Barclay, W. 2002. Fermentation process for producing long chain omega-3 fatty acids with euryhaline microorganisms. In USPTO 6,451,567 B1. Omegatech, Inc., USA.
2. Barclay, W. 2003. Process for the heterotrophic production of microbial products with high concentrations of omega-3 highly unsaturated fatty acids. In US Publ. US20030138477. USA.
3. Bracco, U. and R. Decekbaum. 1992. Polyunsaturated fatty acids in human nutrition. Raven Press, New York, N.Y.
4. Connor, W. E. 2000. Importance of n-3 fatty acids in health and disease. Am J Clin Nutr 71:171 S-175S.
5. Fomuso, L. B., M. Corredig and C. C. Akoh. 2002. Effect of emulsifier on oxidation properties of fish oil-based structured lipid emulsions. J Agric Food Chem 50:2957-2961.
6. Frankel, E. N., T. Satué-Gracia, A. S. Meyer and J. B. German. 2002. Oxidative Stability of Fish and Algae Oils Containing Long-Chain Polyunsaturated Fatty Acids in Bulk and in Oil-in-Water Emulsions. J Agric Food Chem 50:2094-2099.
7. Horrocks, L. A. and Y. K. Yeo. 1999. Health benefits of docosahexaenoic acid (DHA). Pharmacol Res 40:211-225.
8. Kohn, G., W. Banzhaf and J. Abril. 2002. Production and use of a polar lipid-rich fraction containing Omega-3 and/or Omega-6 highly unsaturated fatty acids from microbes, genetically modified plant seeds and marine organisms. Iii PCT Publ No. WO 02092540. Martek Biosciences Boulder Corp.
Kremer, J. M. 2000. n-3 fatty acid supplements in rheumatoid arthritis. Am J Clin Nutr 71:349 S-351S.
10. Kyle, D. J. and C. Becker. 2000. Infant formulas and other food products containing phospholipids. In PCT Publ. WO0054575. Martek Biosciences, USA.
11. Kyle, D. J., S. Reeb and V. Sicotte. 1998. Dinoflagellate biomass, methods for its production, and compositions containing the same. In U.S. Pat. No. 5,711,983. Martek Biosciences.
12. Kyle, D. J., S. E. Reeb and V. J. Sicotte. 1998. Dinoflagellate biomass, methods for its production, and compositions containing the same. InMartek Biosciences Corporation.
13. Lewin, J. and J. A. Hellebust. 1970. Heterotrophic nutrition of the marine pennate diatom, Cylindrotheca fusiformis. Can J Microbiol 16:1123-1129.
14. Radmer, R. 1990. Photobioreactor. InMartek Biosciences Corp.
15. Rosowski, J. R. and B. C. Parker. 1971. Selected Papers in Phycology. Phycological Society of America, Inc.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

What is claimed is:

1. A composition, comprising crude algal extract and lecithin.

2. A composition of claim 1, wherein amount of lecithin ranges from about 4 to about 5 percent by weight of algal extract.

3. The composition of claim 1, wherein the crude algal extract is extracted from an alga grown by fermentation.

4. The composition of claim 3, wherein the said alga is a dinoflagellate.

5. The composition of claim 4, wherein the dinoflagellate is Crypthecodinium.

6. The composition of claim 3, wherein the said alga is a Chytrid.

7. The composition of claim 6, wherein the Chytrid is chosen from Schizochytrium, Tharaustochytrium and Ulkenia.

8. The composition of claim 1, wherein the crude algal extract is extracted from green algae.

9. The composition of claim 8, wherein the green alga is chosen from Nannochloris, and Nannochloropsis.

10. The composition of claim 1, wherein the crude algal extract is extracted from Chrysophytes.

11. The composition of claim 10, wherein the Chrysophyte is Isochrysis.

12. The composition of claim 1, wherein the crude algal extract is extracted from diatoms.

13. The composition of claim 12, wherein the Diatom is chosen from Cyclotella, Navicula, Tetraselmis and Cylindrothecia.

14. The composition of claim 1, wherein the crude algal extract is extracted from Phaeophytes.

15. The composition of claim 14, wherein the Phaeophtes are chosen from Laminaria and Ulva.

16. A composition, comprising algal extract and lecithin.

17. A composition of claim 16, wherein amount of lecithin ranges from about 4 to about 5 percent by weight of algal extract.

18. The composition of claim 16, wherein the algal extract is extracted from an alga grown by fermentation.

19. The composition of claim 18, wherein the said alga is a dinoflagellate.

20. The composition of claim 19, wherein the dinoflagellate is Crypthecodinium.

21. The composition of claim 18, wherein the said alga is a Chytrid.

22. The composition of claim 21, wherein the Chytrid is chosen from Schizochytrium, Tharaustochytrium and Ulkenia.

23. The composition of claim 16, wherein the algal extract is extracted from green algae.

24. The composition of claim 23, wherein the green alga is chosen from Nannochloris and Nannochloropsis.

25. The composition of claim 16, wherein the algal extract is extracted from Chrysophytes.

26. The composition of claim 25, wherein the Chrysophyte is Isochrysis.

27. The composition of claim 16, wherein the algal extract is extracted from diatoms.

28. The composition of claim 27, wherein the Diatom is chosen from Cyclotella, Navicula, Tetraselmis and Cylindrothecia.

29. The composition of claim 16, wherein the algal extract is extracted from Phaeophytes.

30. The composition of claim 29, wherein the Phaeophtes are chosen from Laminaria, and Ulva.

31. A composition, comprising biomeal and lecithin.

32. The composition of claim 31, wherein amount of lecithin ranges form about 4 to about 5 percent by weight of biomeal.

33. The composition of claim 31, wherein the biomeal is extracted from an alga grown by fermentation.

34. The composition of claim 33, wherein the said alga is a dinoflagellate.

35. The composition of claim 34, wherein the dinoflagellate is Crypthecodinium.

36. The composition of claim 33, wherein the said alga is a Chytrid.

37. The composition of claim 36, wherein the Chytrid is chosen from Schizochytrium, Tharaustochytrium and Ulkenia.

38. The composition of claim 31, wherein the biomeal is extracted from green algae.

39. The composition of claim 38, wherein the green alga is chosen from Nannochloris and Nannochloropsis.

40. The composition of claim 31, wherein the biomeal is extracted from Chrysophytes.

41. The composition of claim 40, wherein the Chrysophyte is Isochrysis.

42. The composition of claim 31, wherein the biomeal is extracted from diatoms.

43. The composition of claim 42, wherein the Diatom is chosen from Cyclotella, Navicula, Tetraselmis and Cylindrothecia.

44. The composition of claim 31, wherein the biomeal is extracted from Phaeophytes.

45. The composition of claim 44, wherein the Phaeophtes are chosen from Laminaria and Ulva.

46. A process of producing a food product comprising the addition of the composition of any of claims 1-45 to that food product.

47. The process of claim 46 wherein the food product is intended for consumption by humans.

48. The process of claim 46 wherein the food product is intended for consumption by non-human animals.

49. A process for the stabilization of an algal biomass by the addition of the crude algal extract of any of claims 1-15.

47. A process for the stabilization of an algal biomass by the addition of algal extract of any of claims 16-30.

48. A process for the stabilization of an algal biomass by the addition of biomeal of any of claims 31-45.
